SEQUENCE LISTING (79 sequences)

SEQ ID NO: 1 - protein, 4 aa, Artificial Sequence
  Modified Amino Acids 293-296 of Members of the Genus Metapneumovirus
RRRR

SEQ ID NO: 2 - protein, 4 aa, Artificial Sequence
  Modified Amino Acids 293-296 of Members of the Genus Metapneumovirus
RRRK

SEQ ID NO: 3 - protein, 4 aa, Artificial Sequence
  Modified Amino Acids 293-296 of Members of the Genus Metapneumovirus
RRKK

SEQ ID NO: 4 - protein, 4 aa, Artificial Sequence
  Modified Amino Acids 293-296 of Members of the Genus Metapneumovirus
RKKK

SEQ ID NO: 5 - protein, 4 aa, Artificial Sequence
  Modified Amino Acids 293-296 of Members of the Genus Metapneumovirus
KKKK

SEQ ID NO: 6 - protein, 4 aa, Artificial Sequence
  Modified Amino Acids 293-296 of Members of the Genus Metapneumovirus
KKKR

SEQ ID NO: 7 - protein, 4 aa, Artificial Sequence
  Modified Amino Acids 293-296 of Members of the Genus Metapneumovirus
KKRR

SEQ ID NO: 8 - protein, 4 aa, Artificial Sequence
  Modified Amino Acids 293-296 of Members of the Genus Metapneumovirus
KRRR

SEQ ID NO: 9 - protein, 4 aa, Artificial Sequence
  Modified Amino Acids 293-296 of Members of the Genus Metapneumovirus
SKKK

SEQ ID NO: 10 - protein, 4 aa, Artificial Sequence
  Modified Amino Acids 293-296 of Members of the Genus Metapneumovirus
SRRR

SEQ ID NO: 11 - protein, 4 aa, Artificial Sequence
  Modified Amino Acids 293-296 of Members of the Genus Metapneumovirus
SKKR

SEQ ID NO: 12 - protein, 4 aa, Artificial Sequence
  Modified Amino Acids 293-296 of Members of the Genus Metapneumovirus
SKRR

SEQ ID NO: 13 - protein, 4 aa, Artificial Sequence
  Modified Amino Acids 293-296 of Members of the Genus Metapneumovirus
SRRK

SEQ ID NO: 14 - protein, 4 aa, Artificial Sequence
  Modified Amino Acids 293-296 of Members of the Genus Metapneumovirus
SRKR

SEQ ID NO: 15 - protein, 4 aa, Artificial Sequence
  Modified Amino Acids 293-296 of Members of the Genus Metapneumovirus
SKRK

SEQ ID NO: 16 - protein, 4 aa, Artificial Sequence
  Modified Amino Acids 293-296 of Members of the Genus Metapneumovirus
KGKK

SEQ ID NO: 17 - protein, 4 aa, Artificial Sequence
  Modified Amino Acids 293-296 of Members of the Genus Metapneumovirus
KGRR

SEQ ID NO: 18 - protein, 4 aa, Artificial Sequence
  Modified Amino Acids 293-296 of Members of the Genus Metapneumovirus
KGRK

SEQ ID NO: 19 - protein, 4 aa, Artificial Sequence
  Modified Amino Acids 293-296 of Members of the Genus Metapneumovirus
KGKR

SEQ ID NO: 20 - protein, 4 aa, Artificial Sequence
  Modified Amino Acids 293-296 of Members of the Genus Metapneumovirus
RGKK

SEQ ID NO: 21 - protein, 4 aa, Artificial Sequence
  Modified Amino Acids 293-296 of Members of the Genus Metapneumovirus
RGRR

SEQ ID NO: 22 - protein, 4 aa, Artificial Sequence
  Modified Amino Acids 293-296 of Members of the Genus Metapneumovirus
RGRK

SEQ ID NO: 23 - protein, 4 aa, Artificial Sequence
  Modified Amino Acids 293-296 of Members of the Genus Metapneumovirus
RGKR

SEQ ID NO: 24 - protein, 4 aa, APV-virus type A
RKEK

SEQ ID NO: 25 - protein, 4 aa, APV-virus type B
RHER

SEQ ID NO: 26 - protein, 4 aa, APV-virus type C
SGKD

SEQ ID NO: 27 - protein, 4 aa, Human Metapneumovirus
SGKK

SEQ ID NO: 28 - protein, 539 aa, Human Metapneumovirus
  Modified F protein
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCRRRRGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 29 - protein, 539 aa, Human Metapneumovirus
  Modified F protein
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCRRRKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 30 - protein, 539 aa, Human Metapneumovirus
  Modified F protein
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCRRKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 31 - protein, 539 aa, Human Metapneumovirus
  Modified F protein
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCKKRRGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 32 - protein, 539 aa, Human Metapneumovirus
  Modified F protein
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCKRRRGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 33 - protein, 539 aa, Human Metapneumovirus
  Modified F protein
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSRRRGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 34 - protein, 539 aa, Human Metapneumovirus
  Modified F protein
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCRGRRGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 35 - DNA, 25 bp, Artificial Sequence
  Description of the Artificial Sequence Oligonucleotide
cgagaaaaaaacgcattcaagcagg

SEQ ID NO: 36 - DNA, 22 bp, Artificial Sequence
  Description of the Artificial Sequence Oligonucleotide
gatgtctaggcgaaatccctgc

SEQ ID NO: 37 - DNA, 30 bp, Artificial Sequence
  Description of the Artificial Sequence Oligonucleotide
acgagaaaaaaacgcattcaagcaggttct

SEQ ID NO: 38 - DNA, 27 bp, Artificial Sequence
  Description of the Artificial Sequence Oligonucleotide
gggtatctatgatggtcgacagatgtg

SEQ ID NO: 39 - DNA, 44 bp, Artificial Sequence
  Description of the Artificial Sequence Oligonucleotide
ttaatacgactcactataggaccaatatggaaatatccgatgag

SEQ ID NO: 40 - DNA, 34 bp, Artificial Sequence
  Description of the Artificial Sequence Oligonucleotide
gctaaaaatttgatgaatacggtttttttctcgt

SEQ ID NO: 41 - DNA, 45 bp, Artificial Sequence
  Description of the Artificial Sequence Oligonucleotide
taatacgactcactataggacgagaaaaaaacgcattcaagcagg

SEQ ID NO: 42 - DNA, 21 bp, Artificial Sequence
  Description of the Artificial Sequence Oligonucleotide
ctcaaggttggggggtcgacc

SEQ ID NO: 43 - DNA, 20 bp, Artificial Sequence
  Description of the Artificial Sequence Oligonucleotide
acgggccggccccctgcgtg

SEQ ID NO: 44 - DNA, 44 bp, Artificial Sequence
  Description of the Artificial Sequence Oligonucleotide
aaaagctcttcaattacgagaaaaaaacgcattcaagcaggttc

SEQ ID NO: 45 - DNA, 21 bp, Artificial Sequence
  Description of the Artificial Sequence Oligonucleotide
ctcaaggttggggggtcgacc

SEQ ID NO: 46 - DNA, 22 bp, Artificial Sequence
  Description of the Artificial Sequence Oligonucleotide
ggcatgtacaaagctcgagccc

SEQ ID NO: 47 - DNA, 28 bp, Artificial Sequence
  Description of the Artificial Sequence Oligonucleotide
cattgcaagtgatgttgtcgacattccc

SEQ ID NO: 48 - DNA, 30 bp, Artificial Sequence
  Description of the Artificial Sequence Oligonucleotide
cctcgaaatagggaatctcgagaacatcac

SEQ ID NO: 49 - DNA, 35 bp, Artificial Sequence
  Description of the Artificial Sequence Oligonucleotide
caagcataattgccgcggcgtctcctacagagtgg

SEQ ID NO: 50 - DNA, 35 bp, Artificial Sequence
  Description of the Artificial Sequence Oligonucleotide
ccactctgtaggagacgccgcggcaattatgcttg

SEQ ID NO: 51 - DNA, 25 bp, Artificial Sequence
  Description of the Artificial Sequence Oligonucleotide
gcagggatttcgcgtggacatcttc

SEQ ID NO: 52 - DNA, 27 bp, Artificial Sequence
  Description of the Artificial Sequence Oligonucleotide
caagtgaagatctcgaggcgaaatccc

SEQ ID NO: 53 - DNA, 30 bp, Artificial Sequence
  Description of the Artificial Sequence Oligonucleotide
gatcgtattcaactcgagaacttacctgac

SEQ ID NO: 54 - DNA, 30 bp, Artificial Sequence; identical to SEQ ID NO: 53
  Description of the Artificial Sequence Oligonucleotide

SEQ ID NO: 55 - DNA, 28 bp, Artificial Sequence
  Description of the Artificial Sequence Oligonucleotide
gctatgatcttttgcgtcgacaaagcac

SEQ ID NO: 56 - DNA, 24 bp, Artificial Sequence
  Description of the Artificial Sequence Oligonucleotide
gtacatccagtgctttctcgaggc

SEQ ID NO: 57 - DNA, 26 bp, Artificial Sequence
  Description of the Artificial Sequence Oligonucleotide
cagtgacaggtttttggtcgactatg

SEQ ID NO: 58 - DNA, 22 bp, Artificial Sequence
  Description of the Artificial Sequence Oligonucleotide
gctgagggtgacatactcgagc

SEQ ID NO: 59 - DNA, 22 bp, Artificial Sequence
  Description of the Artificial Sequence Oligonucleotide
gcagccggactcgagctctccc

SEQ ID NO: 60 - DNA, 22 bp, Artificial Sequence
  Description of the Artificial Sequence Oligonucleotide
tagaattcacctgcaggcatgc

SEQ ID NO: 61 - DNA, 21 bp, Artificial Sequence
  Description of the Artificial Sequence Oligonucleotide
gaggatccccgggtaccgagc

SEQ ID NO: 62 - DNA, 29 bp, Artificial Sequence
  Description of the Artificial Sequence Oligonucleotide
cggatatccacaggatccggggataacgc

SEQ ID NO: 63 - DNA, 23 bp, Artificial Sequence
  Description of the Artificial Sequence Oligonucleotide
cgagatcctcgagccggatcctc

SEQ ID NO: 64 - DNA, 42 bp, Artificial Sequence
  Description of the Artificial Sequence Oligonucleotide
ttaatacgactcactatagggacaagtcaaaaatgtctcttg

SEQ ID NO: 65 - DNA, 20 bp, Artificial Sequence
  Description of the Artificial Sequence Oligonucleotide
gtcaaaatgtctcttgaaag

SEQ ID NO: 66 - DNA, 19 bp, Artificial Sequence
  Description of the Artificial Sequence Oligonucleotide
cagggaaagacattgttac

SEQ ID NO: 67 - DNA, 41 bp, Artificial Sequence
  Description of the Artificial Sequence Oligonucleotide
ttaatacgactcactatagggacaagtaacaatgtctttcc

SEQ ID NO: 68 - DNA, 19 bp, Artificial Sequence
  Description of the Artificial Sequence Oligonucleotide
gtaacaatgtctttccctg

SEQ ID NO: 69 - DNA, 34 bp, Artificial Sequence
  Description of the Artificial Sequence Oligonucleotide
gacttgtcccattttttcataactacagatcaag

SEQ ID NO: 70 - DNA, 39 bp, Artificial Sequence
  Description of the Artificial Sequence Oligonucleotide
ttaatacgactcactatagggacaagtgaagatgtctag

SEQ ID NO: 71 - DNA, 19 bp, Artificial Sequence
  Description of the Artificial Sequence Oligonucleotide
gatgtctaggcgaaatccc

SEQ ID NO: 72 - DNA, 28 bp, Artificial Sequence
  Description of the Artificial Sequence Oligonucleotide
gcattgcacttaattattgctgtcaccc

SEQ ID NO: 73 - DNA, 46 bp, Artificial Sequence
  Description of the Artificial Sequence Oligonucleotide
ttaatacgactcactataggaccaatatggaaatatccgatgagtc

SEQ ID NO: 74 - DNA, 39 bp, Artificial Sequence
  Description of the Artificial Sequence Oligonucleotide
gaatgaaaaacaaggaccaatatggaaatatccgatgag

SEQ ID NO: 75 - DNA, 35 bp, Artificial Sequence; identical to SEQ ID NO: 50
  Description of the Artificial Sequence Oligonucleotide

SEQ ID NO: 76 - DNA, 35 bp, Artificial Sequence; identical to SEQ ID NO: 49
  Description of the Artificial Sequence Oligonucleotide

SEQ ID NO: 77 - protein, 6 aa, human RSV
TTNTKE

SEQ ID NO: 78 - protein, 6 aa, Human RSV
TTNIKE

SEQ ID NO: 79 - protein, 6 aa, Bovine RSV
TTDNKE

(19) United States
(12) Patent Application Publication
(10) Pub. No.: US 2006/0002958 A1
(43) Pub. Date: Jan. 5, 2006

(54) Attenuation of metapneumovirus

(75) Inventor: Clive J. Naylor, Neston, Cheshire (GB)

Correspondence Address: PATREA L. PABST;PABST PATENT GROUP LLP, 400 COLONY SQUARE, SUITE 1200, ATLANTA, GA, 30361, US

(73) Assignee: Lohmann Animal Health GmbH & Co. KG, Cuxhaven (DE)

(21) Appl. No.: 10/514,250

(22) PCT Filed: May 16, 2003
(86) PCT No.: PCT/EP03/05187

§ 371 Date: Nov. 12, 2004

(30) Foreign Application Priority Data
May 16, 2002 (DE) 102 21 836.6

(51) Int. Cl. A61K 39/145

(52) U.S. Cl. 424/209.1

(57) ABSTRACT

The present invention relates to a vaccine against members of the genus metapneumovirus or RSV or a virus that comprises a significant genetic homology in the F protein to members of the genus metapneumovirus, whereby the vaccine is an attenuated live vaccine. In particular, the vaccine is directed against metapneumovirus of avian or human origin.

The present invention relates to a vaccine against members of the genus metapneumovirus or RSV, the vaccine being an attenuated live vaccine. In particular, the vaccine is directed against metapneumovirus of avian or human origin.

The present invention further relates to a method for the production of a vaccine which is directed against members of the genus metapneumovirus or RSV. The present invention further relates to an attenuated live vaccine of the genus metapneumovirus. The invention also relates to a host cell comprising such a virus. The present invention is further directed to a specific DNA or cDNA or RNA sequence as well as also to a vector or a plasmid, comprising the DNA, cDNA or RNA sequence.

Finally, the present invention relates to an F protein which is modified, and to the use of the F protein for the production of a vaccine for the prevention of a disorder or a disease produced by viruses belonging to the genus metapneumovirus or RSV.

The paramyxovirus and pneumovirus subfamilies of the paramyxovirus family include several important pathogens for humans and animals. Regarding taxonomy, the pneumoviruses are subdivided into the genera pneumovirus and metapneumovirus. The human respiratorial syncytial virus, the type of the genus pneumovirus, which is also described below, is regarded worldwide as the only and most important trigger of diseases in the lower respiration tract in babies during the early infancy. Other members of the genus pneumovirus include the bovine respiratorial syncytial virus, the ovine respiratorial syncytial virus and the pneumonia virus in mice. The avian pneumovirus APV is also described below and was previously known as the turkey rhinotracheitis virus (TRTV). It is deemed to be an etiological agent of the upper RTI and was previously regarded as the only member of the genus metapneumovirus recently created.

The family of the paramyxoviruses comprises a plurality of various viruses which are involved in various more or less severe disorders or diseases both in humans and animals. The paramyxoviruses contain a single molecule of a single-strain negative RNA as genome and they are all associated with an acute respiratorial disease of the host (Pringe, C. R. (1987) in “Molecular Basis of Virus Disease”, pub. W. C. Russell and J. W. Almond, Society for General Microbiology, vol. 40, pages 91 to 138, Cambridge University Press). Of the many viruses of this family, the respiratorial syncytial virus (RS virus) has the most significant clinical effect in human beings after the measles virus. The human RS virus is the main reason for severe diseases in the lower respiration tract in children which leads annually to epidemics worldwide (Chanock et al., 1991, in “Viral Infections of Humans”, pages 525 to 544, pub. A. S. Evans, Plenum Press). While only a limited nucleotide or amino acid sequence homology is present between the pneumoviruses including the RS virus and the other paramyxoviruses, certain important structural features, such as the main nucleoprotein (N) and the F protein (F) are nevertheless present which are conserved in proteins with similar functions in a broad range of viruses (Barr et al., 1991, J. of Gen. Virol. 72, 677 to 685; Chambers et al., 1990, J. of Gen. Virol. 71, 3075 to 3080). Various RNA synthesis methods are carried out for the paramyxoviruses by the helionucleocapsid complex which is formed by an association of the genomic RNA, the nucleoprotein, a phosphorous protein (P) and the polymerase (L) protein. The function of the nucleoprotein complex depends on the presence of all three proteins interacting with each other and with the genomic RNA and, possibly, with other virus and/or cell components (Barik, 1992, J. of Virol. 66, 6813 to 6818). The maturation of the virion depends on a series of interactions between the ribonucleoprotein complex, the matrix (M) protein and the virus glycoproteins embedded in the modified cell membrane. In pneumoviruses, such as RS, two further proteins, namely a membrane-associated small hydrophobic (SH) protein and a second matrix protein (M2), are also involved in the virion structure and, presumably, the assembly, however in a manner which is not yet understood. The function of the pneumovirus proteins, designated NS1 and NS2, is not known and they do not appear to form a part of the virus particle (Collins, 1991, in “The Paramyxoviruses, pages 103 to 162, pub. D. W. Kingsbury, Plenum Press).

A further economically important virus, belonging to the family of the paramyxoviruses, is the APV virus, which was previously designated TRT virus. The APV virus is a member of the genus metapneumovirus. It triggers turkey rhinotracheitis which is an acute respiratorial disease in turkeys. It was described for the first time at the end of the seventies in South Afrika and later in Europe and other parts of the world. The disease is one of the main reasons for economical losses in the turkey industry during the last years. The serotypes A and B of APV are generally found in Europe, while similar infections were found in Colorado, USA. The APV strain isolated therefrom is the so-called Colorado or C strain. Another APV strain was isolated from ducks in France. Apart from the types A, B and C, further non-A, non-B types of APV presumably exist. Thereby, APV is an avian member of the genus metapneumovirus which was for the first time isolated from birds. The avian metapneumoviruses presumambly also participate in the “swollen head syndrome” which can lead to massive production losses in hens. The avian metapneumovirus (APV) is a pleomorphous RNA virus with a fusion protein (F) localised in the shell, and with spikes consisting of glycoproteins (G). It is designated “metapneumovirus”. The virus does not have hemagglutinating properties. Based on the nucleotides and the estimated amino acid sequence of the glycoprotein (G), the initial APV was classified in two subtypes. APV from South Afrika and Greatbritain had initially the subtype A, and the further European APV strains belonged to the subtype B. As indicated above, additionally to presumably further subtypes which are non-A, non-B, the subtype C is presently also known.

The avian metapneumovirus can be cultivated in tracheal annular cultures having therein a ciliostatic effect. It may further be cultivated in embryonized eggs and in other cell cultures. The American strains were also cultivated in HEF (hen embryofibroblasts), Vero cells and embryonized eggs.

The clinical symptoms of the turkey rhinotracheitis correspond to those of an acute rhinotracheitis infection. The animals were apathetic and exhibited cough, sneezing and head shaking two to three days after infection. If the infection does not become more complicated, the animals are normal again seven to eight days after infection. The clinical symptoms in hens correspond to the symptoms in turkeys, however, as a rule, they are less severe. If E. coli or other bacteria are additionally participating, high losses can result and the actual period of the disease can have a disagreeable duration. However, APV in chickens only rarely leads to “swollen head syndrome” with massive loss. It is probably more important for the continuous loss due to lesser clinical diseases. After the infection by the conventional APV, hens and turkeys create neutralising antibodies in the serum which can be detected by ELISA. These antibodies do not seem to be of significant importance for the control of the rhinotracheitis.

Maternal antibodies are not effective for chicks against an early infection with viral APV. However, circulating antibodies seem to be important for the protection of the gonads in case of infection in elder animals.

Local antibodies seem to have an important protective effect. Virus-specific IgA and IgG antibodies having a virus-neutralising effect appear in the lacrimal fluid after infection with virulent APV. Up to the present, no therapy is known for the treatment of APV infections. With the exception of the USA, attempts are being made to control APV by vaccination.

Another important member of the paramyxoviruses is the newly discovered human metapneumovirus which was isolated from young children having a disease of the respiratory tract. This virus was isolated from 28 young children in the Netherlands and, based on virological data, sequence homology and gene constellation, it was identified as a new member of the genus metapneumovirus (Van Den Hoogen et al., Nature, Medicine, vol. 7, no. 6, June 2001, pages 719 to 724). Until the discovery of this new metapneumovirus, APV was regarded, as described above, as the only member of the genus metapneumovirus recently designated (Virus Taxonomy, Seventh Report of the International Committee on Taxonomy of Viruses (Pub. Van Regenmortel, M. H., Fauquet, C. M. and Bishop, D. H.) 551, 557, 559 to 560 (Academic, San Diego, 2000). In the above article, the authors came, however, to the conclusion, based on a sequence homology and gene constellation, that the human viruses seem to be a new member of the genus metapneumovirus and therefore designated them preliminarily as human metapneumovirus. A genetic homology between the human MPV and e.g. APV in the region of the F proteins has e.g. the high percentage of 80. It is thus assumed that the human metapneumovirus could initially have stemmed from birds and has recently spread to the human population.

As stated above, up to the present no treatment for the above-indicated diseases has been found.

For this reason, attention has been focussed on vaccination. However, no vaccine is up to the present obtainable for the vaccination which could provide a long-term and safe protection against the above diseases associated with viruses, e.g. of the genus metapneumovirus.

Regarding RSV, the development of a vaccine is still an important target in view of the clinical importance of this pathogen. The present approaches involve the use of temperature-sensitive mutants. The attempts to provide a suitable vaccine against the RS virus were focussed on two interesting areas, the specific region of the polymerase (L gene) including the putative ATP binding site and the highly conserved central domain of the polymerase and a mutation in the extra-cellular domain of the fusion proteins (F), from which could be shown that it is also present in an independently derived, coded active attenuated mutant (Connors et al., 1995, Virology 208, pages 478 to 484 and Tolley et al., 1996, Vaccine 14, 1637 to 1646).

The attempt is being made to achieve a control of APV in that e.g. attenuated live vaccines were generated in vitro by the passage of a virulent avian metapneumovirus. It could be shown that approximately 100 passages in tracheal annular cultures did not reduce the virulence, but that 39 passages in the embryoblasts of chickens limited both immunogenity and virulence. The passage in Vero cells reduced virulence to zero, however, essentially maintained immunogenity; acceptable vaccines could be generated thereby.

As a rule, live vaccines are used in about one-day old chicks in the form of a spray or as eye drops. Some producers recommend repeating the immunization with live vaccines once or several times. The live vaccines which are presently on the market are produced from various strains of different kinds and are more or less attenuated. Sometimes, inactivated vaccines are also used for older animals, although, as a rule, they are in this case first vaccinated with a live vaccine.

Up to the present, the live vaccines are of the subtype A or B. The results of various experimental studies show that there is a good cross reactivity between the various strains, however, a homologous protection seems to be more complete. A vaccine against the human metapneumovirus does not yet exist at all since it was discovered only recently. In view of the human metapneumovirus, it was, however, particularly desirable to obtain a vaccine which provides good and effective protection against the virus which leads to severe disease in children.

However, for all vaccines known up to the present it can be proven that they are instable and that they can return to virulence under suitable conditions.

Such properties are naturally undesirable in a vaccine.

It is therefore an object of the present invention to provide a vaccine which can be an attenuated live vaccine and which is directed against members of the virus genus metapneumovirus and RSV.

It is in particular an object to provide an attenuated live vaccine which is effective against pathogens of the member of the genus metapneumovirus, in particular the human metapneumovirus and APV, as well as against RSV.

It is further an object of the present invention to provide a method for the provision of suitable vaccines against the above viruses.

This object is achieved by the subject matter of the independent claims; preferred embodiments are indicated in the dependent claims.

According to claim 1, a vaccine is provided against members of the genus metapneumovirus and RSV as well as against viruses having an important genetic homology in the area of the F protein to members of the genus metapneumovirus, characterized in that it comprises a virus or a part of a virus, the virus being modified in the region aa 293-296 of the amino acid sequence of the F protein (fusion protein) as compared with the wild-type virus, or in a region exhibiting the same function, as e.g. aa 323-328.

A “part of a virus” means that the vaccine must include at least the part of the virus which comprises the modification in region aa 293-296 of the F protein or a functionally similar region and all further components of the virus which are required, so that the virus can act as a vaccine, i.e. generates immunogenity, but is not virulent. The generation of such a virus is a measure within the general special knowledge of a skilled person.

The invention will be further described in accordance with the figures illustrated herein, the figures representing the following:

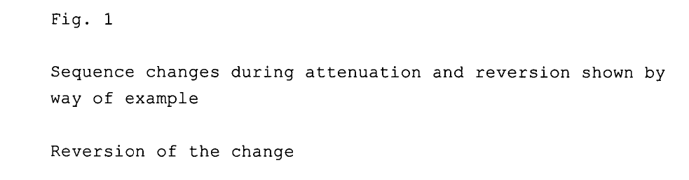

FIG. 1 Sequence Changes by way of example during attenuation and reversion

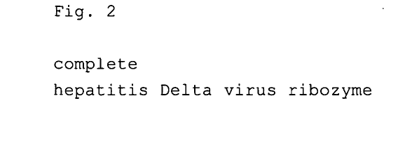

FIG. 2 Cloning strategy

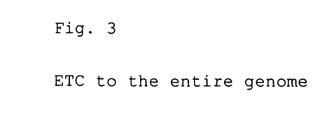

FIG. 3 ETC to the entire genome

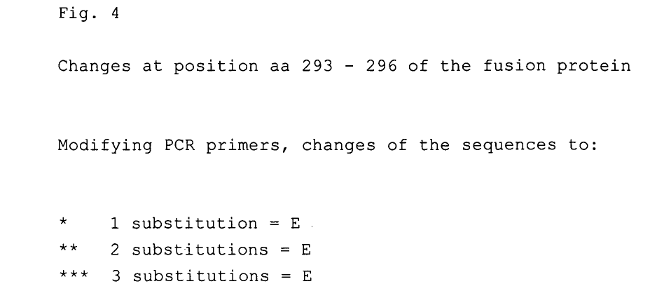

FIG. 4 Changes at positions aa 293-296 of the fusion protein

The usual cleavage position of the fusion protein F, which is e.g. the same for the A type APVs and the other APVs as well as for the other members of the paramyxoviruses, has a motif with basic amino acids which are approximately localized at position aa 99-109, whereby it splits the F protein in F1 und F2 if it is cleaved by proteases, thereby exposing the fusion-related domain. For this reason, the provision of an increased number of basic amino acids in region aa 293-296, which could represent a second cleavage position, or a functionally corresponding position, could lead to a similar cleavage position for proteases.

As a rule, the entire virus is used comprising the modification in region aa 293-296 or in a functionally corresponding region of the amino acid sequence of the F protein. To show the F protein in a typical virus, in particular a metapneumovirus, reference is made to FIG. 1. FIG. 1 shows by way of example the possibility of sequence changes during attenuation and reversion of viruses to virus vaccines and back to virulent viruses.

The abbreviations in FIG. 1 have the following meanings:
N: nucleocapsid; gene: approximately 1,200 bases
P: phosphoprotein; gene: approximately 850 bases
M: matrix; gene: approximately 800 bases
F: fusion; gene: approximately 1,600 bases
M2: 2nd matrix; gene: approximately 600 bases
SH: small hydrophobe; gene: approximately 500 bases
G: glycoprotein; gene: approximately 1,100 bases
L: polymerase; gene: approximately 7,000 bases

It is assumed that the fusion protein is responsible for the fusion with the target membrane. It is assumed that M2 is a transcription-elongation factor which is presumably essentially for virus recovery. It contains a second reading frame which does not seem to be exprimed. The G protein could be responsible for adhesion to a target cell receptor. It is highly glycosylated and highly variable between various strains. The L polymerase relates to the transcription and replication of the genome. N, P and L combine to provide the minimum replication unit which is called nucleocapsid.

The fusion protein is in particular responsible for the fusion of the virus with the target cell. It is translated as F0 and cleaved by cellular proteases at aa positions 99-102 (RRRR) so as to form F1 (containing the fusion-related domain, membrane-bound) and F2 (which remains adhered to F1 after cleavage by disulfide bridges between the cysteine cleavage residues). As explained in detail below, the hypothesis was made that the potential additional cleavage position (aa 293-296 at APV and hMPV and aa 323-328 at RSV) somehow changes the fusion and that this has an influence on the tissue tropism.

The solid-line arrows in FIG. 1 indicate that a sequence change took place during the attenuation at the DNA level, while the dashed-line arrows show that sequence changes took place at the protein level during the attenuation and reversion sequence. The virus arrangement on the left-hand side is that of the wild-type virus. The virus arrangement in the center portion of FIG. 1, represented in white, is that of an attenuated virus, while the virus arrangement on the right-hand side is the arrangement of a virus which has returned to virulence.

It can be derived from FIG. 1 that there is a plurality of possible sequence changes. The sequence changes which can occur during attenuation and reversion and which are represented in FIG. 1 are indicated only as examples.

The comparison of wild-type sequences, attenuated virus and reversed virus clearly shows that there can be a plurality of mutations and a plurality of combinations of mutations occuring during attenuation and reversion.

In accordance with the present invention, it was surprisingly determined that a modification in the region aa 293-296 or in a region having the same functionality leads to a generation of an attenuated virus which can be used as a vaccine against members of the genus metapneumovirus or RSV. The present inventors found a modification in this region will generate an attenuated virus which has lost its virulence capability, while still providing complete immunogenity. Further, the modification in this region will reduce the risk of returning to virulence.

The following Table 1 is presented for a better understanding of the further discussion. It gives a survey of the 20 amino acids, their single letter code (SLC) and their corresponding DNA codons.

TABLE 1

20 Amino acids, their single letter code
(SLC) and their corresponding DNA codons

| | | | |
|---|---|---|---|
| Amino acid | SLC | Type | DNA Codons |

| | | | |
|---|---|---|---|
| Isoleucine | I | | ATT, ATC, ATA |
| Leucine | L | | CTT, CTC, CTA, CTG, TTA, |
| | | | TTG |
| Valine | V | | CTT, GTC, GTA, GTG |
| Phenylalanine | F | | TTT, TTC |
| Methionine | M | | ATG |
| Cysteine | C | S-S | TGT, TGC |
| Alanine | A | | GCT, GCC, GCA, GCG |
| Glycine | G | | GGT, GGC, GGA, GGG |
| Proline | P | | CCT, CCC, CCA, CCG |
| Threonine | T | O glycos | ACT, ACC, ACA, ACG |
| Serine | S | O glycos | TCT, TCC, TCA, TCG, AGT, |
| | | | AGC |
| Tyrosine | Y | | TAT, TAC |
| Tryptophan | W | | TGG |
| Glutamine | Q | | CAA, CAG |
| Asparagine | N | N glycos | AAT, AAC |
| Histidine | H | basic | CAT, CAC |
| Glutamic acid | E | acidic | GAA, GAG |
| Aspartic acid | D | acidic | GAT, GAC |
| Lysine | K | basic | AAA, AAG |
| Arginine | R | basic | CGT, CGC, CGA, CGC, AGA, |
| | | | AGG |
| Stop-Codons | Stop | | TAA, TAG, TGA |

The region aa 293-296 is localized in the F protein, i.e. the fusion protein, of viruses of the genus metapneumovirus and in RSV. The localization of the aa 293-296 region can e.g. be derived from SEQ ID NOS 28 to 34.

The inventors of the present invention could demonstrate that attenuated virus strains could be generated if the codons coding in RSV of the F gene for the amino acids in the aa 293-296 region or in a region having the same functionality, such as aa 323-328, were modified, which maintained their immunogenity but lost their virulence.

It seems that this aa 293-296 region in APV and hMPV or aa 323-328 in RSV was responsible for the virulence change.

The mechanism for this phenomenon is still unclear. Without intending to be bound to this hypothesis, the inventors of the present invention put up the theory that this region is cleaved by serine proteases, but still adhers due to S—S bonds, in a similar manner as is the case with F-1 and F-2. A PAGE of the vaccine shows that, compared with the field virus, there are mobility changes which could be associated with such an event.

The aa 293-296 region in APV and hMPV as well as the aa 323-328 in RSV appears to be essentially hydrophilic, with most of the amino acids being charged (R, K, H, E, D) or having a polarity (S). Only one amino acid, glycine (G), is slightly hydrophobic.

The following amino acid sequences are typical for the different members of the genus metapneumovirus in the region aa 293-296:

(SEQ ID No 24)

| | |
|---|---|
| APV type A: | RKEK, RKKE, REEK |

(SEQ ID No 25)

| | |
|---|---|
| APV type B: | RHER |

(SEQ ID No 26)

| | |
|---|---|
| APV type C: | SGKD |

(SEQ ID No 27)

| | |
|---|---|
| human metapneumovirus: | SGKK |

The following amino acid sequences are typical for the different members of RSV in aa 323-328:

| | |
|---|---|
| 1: TTDNKE | (SEQ ID No 79) |
| 2: TTNIKE | (SEQ ID No 78) |
| 3: TTNTKE | (SEQ ID No 77) |

When reference is made in the following to the human metapneumovirus, that human metapneumovirus is concerned as defined in accordance with the article by Van den Hoogen et al., Nature, Medicine, vol. 7, no. 6, June 2001, pages 719 to 724. It seems to be probable that the hydrophilic region is needed for imparting function and structure to the function protein. Thus, the presence of basic amino acids appears to facilitate the cleavage by serine proteases; accordingly, a suitable modification of the four amino acids in the region aa 293-296 in APV or hMPV as well as a suitable modification of aa 323-328 in RSV seems to have a universally attenuating effect.

According to a preferred embodiment, the above-described virus is an attenuated live virus. Attenuated live viruses are known in the art and their production can be accomplished in a simple manner by a skilled person. According to the present invention, the attenuated live virus must include a modification in region aa 293-296 of the amino acid sequence of the fusion proteins as compared with the wild type or in a region having the same functionality, such as e.g. aa 323-328 in RSV. The attenuation of the virus is obtained by modification in the above region(s). Additionally, known attenuation methods can be used which are already known for these viruses.

Preferably, the modification comprises a stabilization of the region aa 293-296 or a region having the same functionality as aa 323-328 of RSV in the virus.

The designation “stabilization” is meant to decribe a situation in which the amino acids are coded by codons at position aa 293-296 or in a region having the same functionality as aa 323-328 in RSV, which cannot easily return to the wild type. Such a stabilization is preferably obtained by a substitution of codons coding for amino acids in the region, by codons, which need more mutations to return to the wild type. Such a stabilization is e.g. demonstrated as follows:

It is the object of the present example to substitute the amino acid glutamic acid (E) by a basic amino acid. Glutamic acid is coded by the DNA codon GAA or GAG. If the amino acid lysine (K) is selected for the substitution of glutamic acid, the following situation results: Lysine is coded by the DNA codon AAA or AAG. If the DNA codon AAA is selected for lysine, a mutation is required to return to the DNA codon GAA, i.e. glutamic acid. The same applies if the DNA codon AAG is selected which requires one mutation to return to the DNA codon GAG which, in turn codes for glutamic acid. If, for example, the DNA codon CGT (which codes for the basic amino acid arginine (R)) is however selected, three mutations are necessary if the DNA codons GAA or GAG are to be obtained again which code for glutamic acid. Thus, due to the selection of the codon CGT (for arginine) as a substitution for the codon for glutamic acid, more mutations are required to return to the glutamic acid DNA codon, and a higher stabilization will therefore be achieved.

For this reason, in accordance with a futher preferred embodiment, the stabilization is obtained by substitution of codons coding for the amino acid in the region, preferably the acidic amino acids in this region, by codons mutating at a lesser great likelihood to a codon coding for glutamic acid. The inventors of the present invention have found that the presence of glutamic acid in the region aa 293-296 or in a region of the same functionality, such as e.g. aa 323-328 in RSV of the F protein, reduces or prevents attenuation and enhances virulence of viruses.

No prior art document provides any hint which relates to this discovery. Like other acidic amino acids, glutamic acids also seem to contribute to the virulence of the virus when they are located in the region aa 293-296 of the F protein or in a region having the same functionality, such as aa 323-328 in RSV. As stated above, this enhancement of the virulence with a simultaneous attenuation or elimination of the attenuation could be ascribed to a situation in which hydrophilic regions are required to give the F protein a functional structure, while the presence of basic amino acids would promote a serine protease cleavage.

For this reason, according to a further preferred embodiment, the modification in region aa 293-296 of the F protein of viruses or in a region of the same functionality, such as aa 323-328 in RSV, preferably of the genus metapneumovirus, comprises the substitution of at least one non-basic amino acid with a basic amino acid. More preferred, at least two non-basic amino acids are substituted by basic amino acids. Still more preferred, at least three non-basic amino acids are substituted by basic amino acids. According to a particularly preferred embodiment, the amino acids in the region aa 293-296 of the F protein of hMPV or APV are modified such that all four amino acids are basic amino acids.

According to a further preferred embodiment of the present invention, all six amino acids of the region aa 323-328 in RSV are basic amino acids.

According to an alternative preferred embodiment, the modification can comprise the addition of at least one amino acid. This addition of at least one amino acid can be carried out in addition to the above-indicated substitution. Preferably, the addition of at least one amino acid means the addition of at least one basic amino acid, preferably arginine, lysine and/or histidine, particularly preferred is arginine or lysine.

According to a further alternative, the modification can also comprise the deletion of at least one amino acid. This at least one deleted amino acid is preferably an acidic amino acid.

According to a particularly preferred embodiment, the modification comprises the substitution of at least one glutamic acid residue by at least one basic amino acid. The basic amino acid is preferably selected from the following group consisting of arginine, lysine and/or histidine. Especially preferred is the use of arginine and/or lysine.

According to a particularly preferred embodiment, the region aa 293-296 of the wild-type virus has a sequence such as represented in one of the SEQ ID NO 24 to 27.

SEQ ID NO 24 e.g. represents the amino acids in region aa 293-296 of the wild-type APV-A-strain NO. 8544, namely
RKEK.

SEQ ID NO 25 e.g. represents the typical amino acids in region aa 293-296 of APV-B viruses, namely
RHER.

SEQ ID NO 26 represents e.g. the typical amino acids in positions aa 293-296 of viruses of the APV-C type, namely
SGKD.

SEQ ID NO 27 e.g. represents the amino acids in positions aa 293-296 of the human metapneumovirus, namely
SGKK.

The designation “wild-type virus”, as used according to the present invention, relates to viruses which do not exhibit mutations in positions 293-296 or in a region of the same functionality (such as e.g. aa 323-328 in RSV) of the F protein. As a rule, such a wild-type virus is a virulent virus. It is however also possible that an already attenuated live virus is used as the wild-type virus of the present invention which is then further attenuated by the modifications in positions aa 293-296 or in a region having the same functionality as aa 323-328 in RSV, of the F protein according to the present invention.

Preferred viruses which can be wild-type viruses along the lines of the present invention are the human metapneumovirus, APV virus of the types A, B, C or non-A, non-B or the RS virus.

The aa 293-296 region of the attenuated virus has preferably a sequence as is shown in one of the SEQ ID NO 1 to 23.

SEQ ID NO 1 to 23 can also be selected to form a component of the region aa 323-328 in the RS virus.

SEQ ID NO 1 is an exemplary amino acid sequence in position aa 293-296, which allows that e.g. an attenuated strain to be used as a vaccine can be provided e.g. for the strain NO. 8544 (APV type A). This SEQ ID NO 1, namely
RRRR
   could e.g. also provide an attenuated human metapneumovirus or an attenuated APV virus of the B- or C-type or of the non-A- non-B-type.

It is possible to provide an attenuated live virus which can be used as a vaccine by modification of the amino acids or the codons coding for the amino acids at positions aa 293-296 or in a region having the same functionality, such as e.g. aa 323-328 in RSV, of the F proteins as disclosed according to the present invention.

According to a particularly preferred embodiment, the region aa 293-296 of the wild-type virus has the amino acid sequence as represented in SEQ ID NO 24, the region of the attenuated virus having a sequence as shown in SEQ ID NO 1. Such an embodiment would provide an attenuated live virus for the APV type A, e.g. strain NO. 8544.

In accordance with a further preferred embodiment of the present invention, the region aa 293-296 of the wild-type virus has the sequence represented in SEQ ID NO 27, with the region of the attenuated virus having the sequence represented in SEQ ID NO 1, 2, 10 or 21. Such a modification in region aa 293-296 would e.g. provide an attenuated live virus for the human metapneumovirus. The preferred sequences, as represented in SEQ ID NO 1 to 23, could also comprise one or more histidines as a substitution either for lysine or arginine. The above-indicated is mutatis mutandis also applicable for the region aa 323-328 in RSV.

As described above, the present modification in region aa 293-296 or in a region of the same functionality as that of aa 323-328 in RSV of the F protein will lead to a provision of attenuated live viruses, e.g. of the genus metapneumovirus or RSV, which have the common feature of an F protein having substantially a genetic homology between the sequences of the respective F proteins in these viruses.

According to a particularly preferred embodiment, the vaccine produced in accordance with the present invention is effective against the human metapneumovirus. Further preferred is the metapneumovirus avian metapneumovirus, in particular APV.

The present invention is not limited to metapneumovirus, but is applicable for all viruses which have a clear genetic homology, e.g. higher than 50%, preferably higher than 65%, and, particularly preferred, higher than 75% sequence homology, based on sequence homology studies under highly stringent conditions, in the area of the F protein with members of the genus metapneumovirus, in particular with APV or human metapneumovirus.

The attenuated live virus is preferably formulated with a suitable auxiliary agent and/or carrier and/or adjuvant. According to a preferred embodiment, the virus is an attenuated live virus which is formulated with a suitable amount of interleukin-6 (IL-6). The formulation with interleukin-6 is especially preferred if the virus is an avian metapneumovirus.

In accordance with a further embodiment, the virus is an attenuated virus and is formulated with a suitable amount of interleukin-12 (IL-12) and/or interleukin-18 (IL-18). If the virus is a human metapneumovirus or RSV, the attenuated virus is preferably formulated with interleukin-12 and/or interleukin-18.

The designation “a suitable amount” in the context of the present invention designates an amount which provides the desired effect if formulated with the attenuated virus according to the present invention, but does not adversely affect the usability of the attenuated live virus as a vaccine.

Suitable amounts can be determined by a skilled person and will depend on the attenuated virus used and on the subject to be treated, e.g. in view of age, weight, condition of the body and the disease to be treated.

A method for the production of a vaccine according to the present invention directed e.g. against members of the genus metapneumovirus comprises the following steps:
a) providing a virulent virus against which a vaccine is to be developed,
b) providing a modification in the nucleic acid sequence coded for the region aa 293-296 or for a region having the same functionality, such as aa 323-328 in RSV, of the F protein of the virus, and
c) obtaining an attenuated live virus comprising the above modification.

The modification preferably relates to a modification as defined above, in particular according to the following claims 2 to 16.

Further preferred, the virus is a virus selected from the group as defined above, in particular according to the following claims 17 to 19.

The modification indicated above in step b) particularly relates to the region aa 293-296 if APV or hMPV is concerned, and relates to region aa 323-328 if RSV is concerned.

Further, the provision of a modification is preferably obtained as follows:
i) production of a total-length DNA copy of the viral genome of the virus against which a vaccine is to be developed,
ii) provision of copies of the total-length DNA by ligation of partial-length PCR products introducing a change in the region aa 293-296 or in a region having the same functionality such as aa 323-328 in RSV,
iii) virus recovery from the total-length DNA copies by use of e.g. chicken pox T7 polymerase recombinant or cellular ribosomal pol 1 RNA polymerase.

The specific methods which are used so as to introduce mutations at a specific position into a genome are certainly known to the skilled person and/or the common methods can be applied here.

Alterations are carried out in the fusion protein sequence e.g. by PCR amplification using primers which have been changed as compared with the original sequence. The sequence of primers 3.82 Sst neg and 3.82 Sst pos follows the sequence of the embodiment, exept for the substitution of agg aaa aag aaa by agg aga cgc cgc. Two PCRs, one on the side of the leader to 3.82 Sst neg and the other from 3.82 Sst pos to a position downstream (trailer direction), are carried out. The PCR of LTZ 3.1Xho+ to 3.82Sst− (designated 3a) will comprise the changed sequence (change of aa, coding to RRRR) and will additionally contain a Sst11 RE position (together with the adjacent gg, directly upstream of this sequence, [Sst11 recognition position ccgcgg]). The PCR between the primers 3.82 Sst11 pos and LTZ 4.6 Sal− (designated as 3b) generates the same changes in the adjacent fragment, thus, if the two PCR products are cut with Sst11 and ligated to each other, the product will have the original LTZ 3.1Xho+ to LTZ 4.6 Sal-sequence, except for the above-indicated change.

In practice, 3a is initially (bluntly) cloned, then—after checking the sequence—PCR 3b is added (after both the plasmid and 3b were cut with Sst11 and Sal1).

The method may also be used for much longer PCRs using the same primers (3.82 Sst neg and 3.82 Sst pos) and its ligation will result in DNA which can be cut with Sst11 and than be ligated with each other in order to be directly utilized for virus recovery, whereby any cloning stages are prevented. This approach should allow the production of rapidly attenuated viruses either from field isolates or RNA extracts.

FIG. 2 and FIG. 3 indicate a preferred cloning strategy. The genome fragments were initially cloned TWF 18 (LTZ T7 1 to LTZ 9+10 HDVR). Each cloned area (starting with LTZ T7 1 and ending with LTZ 9+10 HDVR) was digested with Xho1 and Sal1, then each was indivudually sequentially cloned in CTPE. In each step, the ligation of the cut Sal1− (plasmid) and Xho1− (leader end of the LTZ fragment) positions of the fragment led to the leader to the intragenome sequence which is resistent against both enzymes, while a combination of two Sal1 cut ends at the trailer end ensured that Sal1 could still be present so as to be able to accept the next fragment. Thus, after the addition from each genome area and the subsequent cloning, the plasmid was again digested with Insert Sal1 and the next area (cut from TWF with Xho1 and Sal1) was cloned thereinto.

According to the present invention, it is also possible to provide a vaccine comprising a mixture of two or more attenuated live viruses, one or a plurality of the attenuated live viruses being obtained in accordance with the present invention, i.e. a modification in the amino acid sequence at positions aa 293-296 or in a region having the same functionality, such as aa 323-328 in RSV, of the F protein.

The present invention also relates to an attenuated live virus which belongs to the genus metapneumovirus or RSV or a virus having a significant genetic homology in the F protein with viruses of the genus metapneumovirus, which is characterized in that it comprises a modification in the region aa 293-296 or in a region having the same functionality, such as e.g. aa 323-328 in RSV, of the F protein. Both the modification as well as the virus are as defined above.

The present invention also relates to a host cell comprising a virus which is attenuated by modification as described above.

According to a further preferred embodiment, the present invention also relates to a DNA or cDNA sequence, as defined in one of the SEQ ID NO 28 to 34. All these sequences 28 to 34 are total-length sequences of the F protein of human metapneumovirus, comprising a suitable modification in region aa 293-296 which provide an attenuated human live metapneumovirus.

The present invention also relates to RNA sequences corresponding to the DNA sequences, as defined in SEQ ID NO 28 to 34.

The present invention also relates to a vector as well as to a plasmid comprising the DNA, cDNA or RNA sequence, as described above.

The present invention further relates to an attenuated live virus which is obtainable by the method as described above.

The present invention also relates to an F protein of a member of the genus metapneumovirus or RSV or a virus sharing a significant genetic homology in the F protein area with a member of the genus metapneumovirus and which is characterized in that it comprises a modification or modifications, such as defined above, i.e. a modification (modifications) of the amino acids at the positions 293-296 or in a region having the same functionality, such as e.g. aa 323-328 in RSV. The modification (modifications) which may be contained in the F protein according to the present invention are defined above.

Finally, the present invention also relates to the use of the F protein, as defined above, or an attenuated live virus, as defined above, for the production of a vaccine for the prevention of a disorder or a disease produced by a virus, as defined above.

The F protein or the attenuated live virus of the present invention can be used for the production of a vaccine for the prevention of a disorder or a disease being triggered by one of the following viruses:
APV type A, B, C or non-A, non-B
human metapneumovirus
RS virus.

The invention is described in accordance with the following examples.

The examples are intended to describe the invention in more detail without limiting the scope of the invention to the specific examples.

### EXAMPLES

1. Production of a Total-Length DNA Copy of a Viral Genome

RNA was extracted from an APV strain LTZ 1 multiplied in Vero cells (German field isolate, collected from LTZ) using Qiagen Rneasy Kits (Quiagen Ltd., Crawley, UK). The RNA was reversely transcribed (Superskript II reverse Transkriptase (Invitrogen Ltd., Paisley, UK)), at 42° C., 1,5 hours, from a viral leader using the primer APV-Lead (5′CGAGAAAAAAACGCATTCAAGCAGG3′) (SEQ ID NO 35) and M2Start+(5′GATGTCTAGGCGAAATCCCTGC 3′)(SEQ ID NO 36), and the cDNA of the leader and trailer areas was repeated 11 times in two 12-cycle PCRs (94° C. 5 sec., 60° C. 20 sec., 68° C. 6 min. [increasing 30 sec. per cycle after cycle 5], amplified with Bio-X-Act DNA polymerase (Bioline, London, UK)). The leader area PCR used the APV lead primed RT reaction as a matrix and PCR primers were APV-Lead ext (5′ACGAGAAAAAAACGCATTCAAGCAGGTTCT3′)(SEQ ID NO 37) and LTZ 8.2 sal neg (5′GGGTATCTATGATGGTCGACAGATGTG3′) (SEQ ID NO 38). The trailer area used M2Start+primed RT-reaction as a matrix and the PCR primers were LT7 (5′TTAATACGACTCACTATAGGACCAATATGGAAATATCCGATGAG3′)(SEQ ID NO 39) and APV trail ext (5′GCTAAAAATTTGATGAATACGGTTTTTTTCTCGT3′)(SEQ ID NO 40). The 2 PCRs acted as matrices for further 30 cycles PCRs (94° C. 5 sec., 60° C. 20 sec., 68° C. 2 min. [increasing 10 sec. per cycle after cycle 5], repeated 29 times) using pfu (Stratagene, Amsterdam, NL), which amplified the entire genome in 8 areas, designated as LTZ 1 to 10. PCR primers include the sequence modifications which introduced the restriction endonuclease-recognition positions or other changes at the DNA extremities and 3′-extremities, while, with the exception of LTZ 3, the coded protein sequence remained unchanged. The T7 promoter was added to the viral leader sequence in LTZ 1T7, a shortened form of the human RNA polymerase-1-promoter (pol 1) was added to the viral leader sequence in LTZ 1 pol, the area which coded for aa 293-296 of the fusion proteins was changed such that aa RKKK became RRRR in LTZ 3, and in LTZ 10 HDVR the hepatitis Delta virus ribozyme was added to the viral trailer area (LTZ 9+10). The changes in the F protein gene sequence increased the number of mutations which were required so that the sequence could mutate to a sequence which coded for acidic amino acids, as is represented in detail in FIG. 4.

TABLE 2

Sequence of the PCR primers used for
generating Sall/Xhol-flanked genome areas

| | | | SEQ |
|---|---|---|---|
| LTZ | | | ID |
| area | Primer | Sequence 5′-3′ | NO |
| T7 1 | T7 APV | TAA TACGACTCACTATAGGACGAGAAAAAAACG | 41 |
| | lead 1 | CATTCAAGCAGG | |
| | LTZ 1.1 | CTC AAG GTT GGG GGG TCG ACC | 42 |
| | sal− | | |
| Pol | pol 1 | ACG GGC CGG CCC CCT GCG TG | 43 |
| 1 | start+ | | |
| | Lead Sap | AAAAGCTCTTCAATTACGAGAAAAAAACGCATTC | 44 |
| | 1 | AAGCAGGTTC | |
| | LTZ 1.1 | CTC AAG GTT GGG GGG TCG ACC | 45 |
| | sal− | | |
| 2 | LTZ 1.1 | GGC ATG TAC AAA GCT CGA GCC C | 46 |
| | Xho+ | | |
| | LTZ 3.1 | CAT TGC AAG TGA TGT TGT CGA CAT | 47 |
| | sal− | TCC C | |
| 3 | LTZ 3.1 | CCT CGA AAT AGG GAA TCT CGA GAA | 48 |
| | Xho+ | CAT CAC | |
| | F 3.82 | CAA GCA TAA TTG CCG CGG CGT CTC | 49 |
| | Sst− | CTA CAG AGTGG | |
| | F 3.82 | CCA CTC TGT AGG AGACGC CGC GGC AAT | 50 |
| | Sst+ | TAT GCT TG | |
| | LTZ 4.6 | GCA GGG ATT TCG CGT GGA CAT CTT C | 51 |
| | sal− | | |
| 4+5 | LTZ 4.6 | CAA GTG AAG ATC TCG AGG CGA AAT | 52 |
| | Xho+ | CCC | |
| | LTZ 7.6 | GAT CGT ATT CAA CTC GAG AAC TTA | 53 |
| | sal− | CCT GAC | |
| 6+7 | LTZ 7.6 | GAT CGT ATT CAA CTC GAG AAC TTA | 54 |
| | Xho+ | CCT GAC | |
| | LTZ 9.9 | GCT ATG ATC TTT TGC GTC GAC AAA | 55 |
| | sal− | GCA C | |
| 8 | LTZ 9.9 | GTA CAT CCA GTG CTT TCT CGA GGC | 56 |
| | Xho+ | | |
| | LTZ11.9 | CAG TGA CAG GTT TTT GGT CGA CTA TG | 57 |
| | sal− | | |
| 9+10 | LTZ11.9 | GCT GAG GGT GAC ATA CTC GAG C | 58 |
| | Xho+ | | |
| HDVR | HDVR | GCA GCC GGA CTC GAG CTC TCC C | 59 |
| | Xho− | | |

With the exception of LTZ 3, each blunt-ended PCR product was ligated in the purposely constructed plasmid PTWF 18, which was a pUC 18-derived plasmid in which the Sal1-position was changed to EcoR1. The plasmid was copied and changed using a modified primer (p18-eco420-5′ TAG AAT TCA CCT GCA GGC ATG C3′ (SEQ ID NO 60) in a PCR based on pfu (cycle 94° C. 5 sec., 60° C. 20 sec., 68° C. 3 min. [increasing 10 sec. per cycle after cycle 5] 29 times repeated), in another primer p18-400+5′ GAG GAT CCC CGG GTA CCG AGC3′ (SEQ ID NO 61). The PCR products themselves were ligated over night (Bioline QS ligase, over night at 14° C.) and then used so as to transform competent DH5alpha cells (Invitrogen) under standard conditions (LB Agar/Broth, Xgal plates, ampicillin 100 μg/ml, 37° C.). The blue colonies were selected and a specific restriction endonuclease digestion was carried out so as to confirm that two EcoR1 positions were present and that the Sal1 position was deleted.

The LTZ areas (except for T71 and 3) were ligated in the sma1 position of pTWF18 (Bioline QS ligase, over night at 14° C.). The ligation mixtures were used for the transformation of competent DH5alpha cells. For stability considerations, the whole cultivation work of the bacteria with plasmids which contained the LTZ genomes or parts thereof was performed at 30° C. LTZ 3 was cloned in pUC 18 in two halfs. The 5′ half (3a, in anti-genomic sense) was amplified and modified using the primers LTZ 3.1 Xho+ and F 3.82 Sst neg (see Table 2) in a PCR of 30 cycles (94° C. 5 sec., 45° C. 20 sec., 68° C. 2 min. [increasing 10 sec. per cycle after cycle 5] repeated for 29 times) using pfu polymerase. The same was ligated into the Sma1 position of pUC18 and cloned in DH5alpha (pUC18-3a). The orientation was checked by use of a Sst11, EcoR1 double digestion. The 3′ half (3b) was amplified using the primers F 3.82 Sst pos and LTZ 4.6 sal− (see Table 2) and using a cycle of 94° C. 5 sec., 50° C. 20 sec., 68° C. 2 min. [increasing 10 sec. per cycle after cycle 5] repeated for 29 times and using the pfu polymerase. The same was cut with Sst11 and Sal1 and cut pUC18-3a (Bioline QS ligase) was ligated in Sst11 and Sal1. The resulting ligation mixture was used for the transformation of DH5alpha and the resulting clones were designated pLTZ 3 RRRR.

All cloned LTZ areas were sequenced (Imperial College Medical School Service) and, except for the case that the protein sequence was not concerned, the area was recloned if mutations were present.

The low copy plasmid PCTPE was a modification of pOLTV5 (Peeters et al. (1999), J. Virol. 23, 5001-5009) in which the cloning efficiency was amplified by the deletion of HDVR, T7-terminator and the remaining, partially lac z, genome areas using therein a similar approach like that used so as to produce pTWF18. In this case, both PCR primers were modifying (V5 630BE+5′CGG ATA TCC ACA GGA TCC GGG GAT AAC GC3′ (SEQ ID NO 62) and V5 190 Bam-5′ CGA GAT CCT CGA GCC GGA TCC TC3′ (SEQ ID NO 63) and introduced new EcoRV blunt-ended positions, each side flanked by BamH1 positions. All growth media contained kanamycin with 15 μg/ml (Gibco, Invitrogen, Paisley, UK).

The PCR product LTZ T71 was cloned in the EcoRV position of pCTPE, whereby pCTPE-LTZT71 was produced and the entire sequence of the insert was confirmed. It was then digested with Sal1, wherein LTZ2 was ligated and cloned which was cut from pTWF18 (Xho1 Sal1 double digestion) (DH5alpha (Invitrogen), kanamycin plates and kanamycin broth). The orientation was checked by comparing a BamH1 digestion with a BamH1, Sal1 double digestion. The plasmid with the correctly oriented insert (pCTPE-LTZT71,2) was only cut with Sal1 and Xho1- and Sal1-digested LTZ 3 RRRR was added thereto and then ligated in the previous manner. The method was continued in a similar manner from the 5′ end (in anti-genomic sense), until the entire genome was cloned together with the T7 promoter and HDVR, (pCTPE-LTZT7,1,2,3RRRR,4+5, 6+7,8,9+10-HDVR, better PLTZ f1T7 or pLTZf1pol1).

2. Carrier Proteins

Nucleocapsid (N), phospho (P) and matrix 2 (M2) sequences of the strain LTZ1 were copied and cloned. The RNA was extracted (Rnease, Qiagen) which was afterwards reversely transcribed and amplified by RT-PCR (Superskript 2 Invitrogen; BioX-Act, Bioline; cycle of 94° C. 5 sec., 50° C. 20 sec., 68° C. 2 min. [increasing 10 sec. per cycle after cycle 5], 29 times repeated), using therein primers (see below), which introduced the T7-promoter sequence directly before the start codon of each gene and continued further beyond each stop codon.

TABLE 3

Sequence of PCR primers which were used for
multiplying the carrier protein gene

| | | | |
|---|---|---|---|
| Gene | Primer | Sequence 5′-3′ | SEQ ID NO |

| | | | |
|---|---|---|---|
| N | T7-N | TTA ATA CGA CTC ACT ATA GGG | 64 |
| | | ACA AGT CAA | |
| | TGT | CTC TTG | |
| | N | GTC AAA ATG TCT CTT GAA AG | 65 |
| | start+ | | |
| | Pstart− | CAG GGA AAG ACA TTG TTA C | 66 |
| P | T7-P | TTA ATA CGA CTC ACT ATA GGG | 67 |
| | | ACA AGT AAC AAT GTC TTT CC | |
| | Pstart+ | GTA ACA ATG TCT TTC CCT G | 68 |
| | Pstop | GAC TTG TCC CAT TTT TTC ATA | 69 |
| | neg ext | ACT ACA GAT CAA G | |
| M2 | T7-M2 | TTA ATA CGA CTC ACT ATA GGG | 70 |
| | | ACA AGT GAA GAT GTC TAG | |
| | M2start | GAT GTC TAG GCG AAA TCC C | 71 |
| | + | | |
| | M2-1^{st} | GCA TTG CAC TTA ATT ATT GCT | 72 |
| | neg | GTC ACC C | |
| L | T7-L | TTA ATA CGA CTC ACT ATA GGA | 73 |
| | | CCA ATA TGG AAA TAT CCG ATG | |
| | | AGT C | |
| | L start | GAA TGA AAA ACA AGG ACC AAT | 74 |
| | Xho+ | ATG GAA ATA TCC GAT GAG | |

T7-prefixated genes were cloned into the sma1 position of pUC18, using a procedure which was identical to that used for TWF above. PCR products without T7 promoter were cloned into the sma1 position of the pTarget (mammalian expression vector, Promega, Southhampton, UK).

The viral polymerase gene (L) was cloned in areas in the EcoRV position of pCTPE using the sequential preparation which was used for the complete viral genome. The ligation was carried out in the following sequence: T7 L start, LTZ 6+7, LTZ8, LTZ 9+10 in pCTPE. The LTZ T7 L start was a PCR product (30 cycles 94° C. 5 sec., 60° C. 20 sec., 68° C. 2 min. [increasing 10 sec. per cycle after cycle 5], 29 times repeated, using Pfu [Stratagene]), produced of the above-described 12-cycle trailer PCR, using the primer T7-L (Table 3) and LTZ 7.6Xho− (Table 2). The LTZ T7 start was ligated in the CPTE EcoRV position, while the following areas of pTWF18 (Sal1 und Xho1) were cut and ligated in the new unique Sal1 position, which was introduced in each step of the cloning. The orientation was checked in the same manner as for the full-length genome.

The L gene was also cloned in the pTarget in a similar manner as that described for the starter sequence (produced with use of the primer L Start Xho+ (Table 2) and LTZ 7.6Xho− (Table 2)) and no T7 promoter was previously added thereto.

3. Total-Length Copy by PCR Copy

The entire genome with a previously added T7 promoter was copied and multiplied in 3 PCRs (Bioline Bio-X-act, 30 cycles 94° C. 5 sec., 60° C. 20 sec., 68° C. 4 min. [increasing 10 sec. per cycle after cycle 5]), using low copy (12×) PCRs which were previously mentioned as matrices. The leader and central areas used the APV lead ext and LTZ 8.2 sal product, and the trailer area used the LT7 and APV trail ext product.

The primers used were the following: Leader area, T7-APV lead 1 and F 3.82 sst pos (51 CCA CTC TGT AGG AGA CGC CGC GGC AAT TAT GCT TG3′ (SEQ ID NO 75); central area F 3.82 sst neg (5′CAA GCA TAA TTG CCG CGG CGT CTC CTA CAG AGT GG3′) (SEQ ID NO 76) and LTZ 8.2 Sal- and the trailer area used LTZ 8.2 Xho+ and APV trail ext. In this manner, the restriction endonuclease positions of SstII) and Xho/Sal were added at the connecting positions 3826-3831 and 8204-8209, respectively, so as to allow a later ligation of the three areas, and a change of the protein coding to RRRR was further introduced into the F gene at the aa positions 293-296. The areas were cut with SstII (leader area), SstII and Sal1 (central area) and Xho1 (trailer area) and connected using highly concentrated T4 DNA ligase (over night, Fermentas, Germany, 30 U/μl).

4. Virus Recovery

a) Using chicken pox-T7-Polymerasere Combinant

Vero cells (70% confluent) in 35 mm recesses were washed once with 1.0 ml Optimem 1 and then infected with chicken pox-T7-polymerasere combinant having an MOI of 0.2. After an incubation time of 1 hour, the medium was removed and the cells were washed with 1 ml of Optimem 1 and subsequently with 2 ml of Optimem 1.

MEM (5%) FCS was added. A DNA/Fugene 6 (Boehringer Mannheim, Lewes, UK) complex was cloned by mixing 4 cloned carrier protein genes N, P, M2 in pUC18 (0.5 μg each), L in pTWF18 (50 ng), total length genome (1 μg either cloned in pCPTE or as ligated PCR product), and 10 μl of Fugene6 (Boehringer Mannheim) was produced and dissolved in 300 μl of dMEM. After complete mixing, this mixture was added drop by drop to cells while carefully shaking the same.

Five days later, the supernatant was collected, fed through a 0.2 μm filter and used for inoculating fresh Vero cells. CPE was observed and the cells were dyed to detect the presence of TRT antigene using indirect immunofluorescence dyeing. It was confirmed that the virus originated from the total length copy produced, in that PCR copies of the unique RRRR regions of the F gene and other Sal1/Xho1 connection regions were sequenced.

b) Using Cellular Ribosomal Poll RNA Polymerase

Vero cells (70% confluent) or CEFs in 35 mm recesses were washed once with 1.0 ml of Optimem 1, whereupon 2 ml of MEM (5%) FCS were added. The DNA/Fugene 6 (Boehringer Mannheim) complex was produced in that the four cloned carrier protein genes N, P, M2 (0.5 μg each), L (50 μg) in pTarget, pol1 total length genome (1 μg, cloned in pCPTE) and 10 μl of Fugene 6 (Boehringer Mannheim) were mixed and dissolved in 300 μl of dMEM. After complete mixing, this mixture was added drop by drop to cells while carefully shaking the same.

Five days later, the cells were freeze-dried and the clarified supernatants were used for infecting fresh cells. CPE was observed and the cells were dyed to detect the presence of TRT antigene using indirect immunofluorescence dyeing. It was confirmed that the virus originated from the total length copy produced, in that the PCR copies were sequenced from the unique RRRR regions of the F gene and Sal1/Xho1 binding regions.

1. A vaccine against a member of the genus metapneumovirus, respiratorial synctial virus (“RSV”) or a virus which has a significant genetic homology in the F protein area with a member of the genus metapneumovirus, comprising the virus or part of the virus modified in a region corresponding to amino acids 293-296 of the amino acid sequence of the F protein (fusion protein) as compared with the wild-type virus.

2. The vaccine according to claim 1, wherein the virus is a live virus.

3. The vaccine according to claim 1, wherein the virus is an attenuated live virus.

4. The vaccine according to claim 1, wherein the modification comprises a stabilization of the modified region.

5. The vaccine according to claim 4, wherein the modification comprises a substitution of codons coding for amino acids in the region with codons requiring more mutations in order to return to the wild-type virus.

6. The vaccine according to claim 4, wherein the modification comprises a substitution of codons, coding for amino acids in the region with codons which with lesser probability mutate back to a codon which codes for glutamic acid.

7. The vaccine according to claim 1, wherein the modification comprises the substitution of at least one non-basic amino acid by at least one basic amino acid.

8. The vaccine according to claim 1, wherein the modification comprises the addition of at least one amino acid.

9. The vaccine according to claim 1, wherein the modification comprises the deletion of at least one acidic amino acid.

10. The vaccine according to claim 1, wherein the modification comprises the substitution of at least one glutamic acid residue by at least one basic amino acid.

11. The vaccine according to claim 10, wherein at least one basic amino acid is selected from the group consisting of arginine, lysine, and histidine.

12. The vaccine according to claim 11, wherein at least one basic amino acid is arginine lysine.

13. The vaccine according to claim 1, wherein the modified region comprises a sequence selected from the group consisting of SEQ ID NO 24, SEQ ID NO 25, SEQ ID NO 26, and SEQ ID NO 27.

14. The vaccine according to claim 3, wherein the modified region of the attenuated virus consists essentially of a sequence selected from the group consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19. SEQ ID NO 20, SEQ ID NO 21, SEQ ID NO 22, and SEQ ID NO 23.

15. The vaccine according to claim 13, wherein the modified region of the wild-type virus consists essentially of SEQ ID NO 24, and wherein the F protein region of the attenuated virus comprises a sequence as represented in SEQ ID NO 1.

16. The vaccine according to claim 13, wherein the modified region of the wild-type virus consists essentially of SEQ ID NO 27, and wherein the F protein region of the attenuated virus comprises a sequence as is represented in SEQ ID NO 1, 2, 10 or 21.

17. The vaccine according to claim 1, wherein the modification in the modified region is selected to provide a vaccine against APV or hMPV.

18. The vaccine according to claim 1, wherein the metapneumovirus is a human metapneumovirus or an avian metapneumovirus.

19. The vaccine according to claim 1, wherein the metapneumovirus is an avian metapneumovirus.

20. The vaccine according to claim 1, further comprising a auxiliary agent, carrier or adjuvant.

21. The vaccine according to claim 20, wherein the auxiliary agent is selected from the group consisting of interleukin-6 (IL-6), of interleukin-12 (IL-12) and interleukin-18 (IL-18).

22. The vaccine according to claim 21, wherein the virus is an attenuated virus which is formulated with a suitable amount of interleukin-12 (IL-12) or interleukin-18 (IL-18).

23. A method for the production of a vaccine directed against members of the genus metapneumovirus, respiratorial syncytial virus (“RSV”) or a virus which has a significant genetic homology in the F protein area with a member of the genus metapneumovirus, comprising:
a) providing a virulent virus against which a vaccine is to be developed;
b) providing a modification in the nucleic acid sequence coding for the region corresponding to amino acids 293-296 of the F protein, and
c) obtaining an attenuated live virus comprising the modification.

24. (canceled)

25. (canceled)

26. The method according to claim 23, wherein step b comprises:
i) producing a total-length DNA copy of the viral genome of the virus against which a vaccine is to be developed,
ii) providing copies of total-length DNA by litigation of partial lengths of PCR products which introduce a change in modified region, and
iii) recovering the virus from the total-length DNA copies.

27. An attenuated live virus belonging to the genus metapneumovirus or pneumovirus, comprising a modification in the region corresponding to amino acids 293-296 of the F protein.

28. (canceled)

29. (canceled)

30. A host cell comprising a virus according to claim 27.

31. A DNA or cDNA sequence selected from the group consisting of SEQ ID No. 28, 29, 30, 31, 32, 33 and 34.

32. An RNA sequence corresponding to the DNA or cDNA sequence as defined in claim 31.

33. An F protein of a member of the genus metapneumovirus, respiratorial syncytial virus (“RSV”) or of a virus which has a significant genetic homology in the F protein to a member of the genus metapneumovirus, modified in the region corresponding to amino acids 293 to 296.

34. A vector, comprising the DNA, cDNA or RNA sequence according to claims 31 or 32.

35. (canceled)

36. A plasmid, comprising the DNA, cDNA or RNA sequence according to claim 31 or 32.

37. A method of vaccinating an individual for the prevention of a disorder or disease triggered by members of the genus metapneumovirus, respiratorial syncytial virus (“RSV”) or a virus which has a significant genetic homology in the F protein area with a member of the genus metapneumovirus, comprising administering an F protein of a member of the genus metapneumovirus, respiratorial syncytial virus (“RSV”) or of a virus which has a significant genetic homology in the F protein to a member of the genus metapneumovirus, modified in the region corresponding to amino acids 293 to 296, or an attenuated live virus belonging to the genus metapneumovirus or pneumovirus, comprising a modification in the region corresponding to amino acids 293-296 of the F protein.

38. A primer selected from the group consisting, of SEQ ID NO 35, 36, 37, 38, 39, 40, 41, 42, 43, 44, 45, 46, 47, 48, 49, 50, 51, 52, 53, 54, 55, 56 57, 58, 59, 60, 61, 62, 63, 64, 65, 66, 67, 68, 69, 70, 71, 72, 73, 74, 75 and 76.

39. The vaccine according to claim 1, wherein the modified region comprising amino acids 323-328 in RSV.

40. The vaccine according to claim 14, wherein the modified region comprises SEQ ID NO 1.

41. The vaccine according to claim 14, wherein the modified region comprises a sequence selected from the group consisting of SEQ ID NO 1, SEQ ID NO 2, SEQ ID NO 10, and SEQ ID NO 21.
